(19) United States
(12) Patent Application Publication
(10) Pub. No.: US 2012/0244101 A1
(43) Pub. Date: Sep. 27, 2012

(54) Self-Leveling Cosmetic

(75) Inventors: Kelly Hales, Nyack, NY (US); Hossein A. Baghdadi, Portland, OR (US); Derrick B. McKie, Brooklyn, NY (US)

(73) Assignee: AVON PRODUCTS, INC., New York, NY (US)

(21) Appl. No.: 13/513,980

(22) PCT Filed: Nov. 10, 2010
(86) PCT No.: PCT/US10/56113

§ 371 Date: Jun. 5, 2012

Related U.S. Application Data
(60) Provisional application No. 61/287,364, filed on Dec. 17, 2009.

(51) Int. Cl. A61K 8/87; A61Q 1/10; A61K 8/92

(52) U.S. Cl. 424/70.7

(57) ABSTRACT

Cosmetic compositions comprising a Boger fluid are provided as well as methods for using such compositions in cosmetics that exhibit Boger fluid-like characteristics, such as self-leveling. These cosmetics provide for ease of application and aid in achieving a smooth, even, and/or glossy finish on skin and hair.

### CROSS REFERENCE TO RELATED APPLICATIONS

This application claims priority to International Application Serial No. PCT/US10/56113 filed Nov. 10, 2010, which claims priority U.S. Provisional Patent Application Ser. No. 61/287,364 filed Dec. 17, 2009, the contents of which are hereby incorporated by reference in their entirety.

### FIELD OF INVENTION

The present invention relates generally to cosmetic compositions having a unique rheology that provides a self-leveling property to substantially eliminate clumping and surface irregularities when applied to an integument.

### BACKGROUND OF THE INVENTION

A Boger fluid is an elastic fluid with a constant viscosity, meaning that its viscosity is independent of shear rate or nearly so. See James, D. F. (January 2009) Annual Review of Fluid Mechanics; 41: 129-142. Accordingly, over a wide range of shear rates (γ), the viscosity (η) of a Boger fluid remains essentially constant, giving the fluids characteristic rheological properties. Boger fluids generally are dilute polymer solutions, often made with a solvent sufficiently viscous that stresses due to elasticity are measurable. In addition to constant viscosity, a Boger fluid has a non-zero first normal stress difference, making the fluid non-Newtonian.

Boger fluids have been extensively used in the paint and industrial coatings industry. A specific example involves systems using polymers of polyether polyurethane. In the paint industry, one such polymer is known by the trade name Rheolate® 288, and is commercially available from Elementis Specialties, Inc. Rheolate® 288 is suspended in water and butyl carbitol, the latter constituent possibly posing a toxicology issue with respect to human tissues when incorporated in a cosmetic product for topical application, especially in a mascara used proximate to the eyes. Rheolate® 288 has not been used in the cosmetic industry and, moreover, its application to provide self-leveling mascaras has not been recognized.

While consumers seek cosmetics that are easy to apply and that provide a smooth, even appearance to skin and lashes, there remains a need in the cosmetic industry for such products. Accordingly, there is a need in the cosmetic arts for cosmetic compositions having Boger fluid-like properties, where such compositions could facilitate uniform application of the cosmetic to skin, hair or eyelashes. It is therefore an object of the invention to provide compositions and methods addressing these and other needs.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE INVENTION

In accordance with the foregoing objectives and others, it has surprisingly been found that cosmetic compositions comprising polyether polyurethane associative thickeners behave similarly to Boger fluids and are characterized by improved flow and self-leveling properties. When applied to an integument, such as the hair, lashes, skin, or nails, the resulting cosmetic film will self-level, by which is meant that initially existing clumps and surface irregularities will be substantially eliminated within a few minutes (e.g., less than three minutes, less than two minutes, or even less than one minute) or less without requiring action by the user. In particular, the present invention relates to mascara compositions for application to the eyelashes which self-level to provide smooth films coating the shaft of the eyelashes.

One aspect of the invention relates to cosmetic compositions comprising an associative thickener capable of forming a film on the shaft of a keratin fiber as an initial coating, whereafter the coating can self-level to provide a coating having reduced clumping and a smoother surface as compared to the initial coating. In some embodiments, the associative thickener comprises a polyurethane polymer resulting from the reaction of a mixture comprising (i) a polyether polyol component having an average molecular weight between about 500 and about 20,000 g/mol, (ii) a diisocyanate, (iii) a branched, aliphatic C₈₋₃₆ primary alcohol, and (iv) optionally a modifier comprising two groups reactive with said diisocyante and a hydrophobic moiety. In some preferred embodiments, the polyurethane polymer comprises polyether urea polyurethane polymer.

In some embodiments, the composition further comprises one or more wax components in an amount of up to about 35% by weight. In some embodiments, the composition is an aqueous system comprising the polyurethane polymer, propylene glycol and/or water. In some embodiments, the composition is in an oil-in-water or a water-in-oil emulsion. In some embodiments, the polymer is present in the composition in an amount from about 0.01 weight % to about 30 weight %, or from about 0.1 weight % to about 25 weight %, or from about 0.5 weight % to about 20 weight %, or from about 1 weight % to about 15 weight %, or about 2 to about 10 weight %, based on the total weight of the composition. In some particularly preferred embodiments, the composition is an anti-clump mascara. Suitable amounts of the polymer are about 0.1 weight %, or about 0.5 weight %, or about 1 weight %, or about 2 weight %, or about 5 weight %, or about 10 weight %, or about 15 weight %, or about 20 weight %.

Another aspect of the invention relates to methods for using such compositions. In some embodiments, a method is provided for applying a smooth cosmetic film to eyelashes, comprising forming an initial coating on the eyelashes with a composition comprising an associative thickener capable of forming a film on the shaft of a keratin fiber; and allowing the coating to self-level to provide a coating having reduced clumping and a smoother surface as compared to the initial coating. The composition can be used as an anti-clump mascara, that facilitates coating eyelashes smoothly and evenly.

Still another aspect of the invention relates to methods for imparting a self-leveling property to a mascara. In certain embodiments, the method comprises reducing the alcohol content of the mascara to produce a near alcohol-free composition; and adding an associative thickener comprising a polyurethane polymer resulting from the reaction of a mixture comprising (i) a polyether polyol component having an average molecular weight between about 500 and about 20,000 g/mol, (ii) a diisocyanate, (iii) a branched, aliphatic C₈₋₃₆ primary alcohol, and (iv) optionally a modifier comprising two groups reactive with said diisocyante and a hydrophobic moiety, where the polyurethane polymer is added in an amount effective to maintain a constant or near constant viscosity of the near alcohol-free composition over a plurality of shear rates. In some preferred embodiments, the polyurethane polymer comprises a polyether urea polyurethane polymer, and/or is in an amount from about 0.01 weight % to about 30 weight %, or from about 0.1 weight % to about 25 weight %, or from about 0.5 weight % to about 20 weight %, or from about 1 weight % to about 15 weight %, or about 2 to about 10 weight %, based on the total weight of the composition. Suitable amounts of the polymer are about 0.1 weight %, or about 0.5 weight %, or about 1 weight %, or about 2 weight %, or about 5 weight %, or about 10 weight %, or about 15 weight %, or about 20 weight %. In some preferred embodiments, the near-alcohol free composition further comprises at least one wax component.

A method of manufacturing a cosmetic composition is also provided. The method generally comprises incorporating into said cosmetic composition an aqueous polyol dispersion of a polyurethane polymer resulting from the reaction of a mixture comprising (i) a polyether polyol component having an average molecular weight between about 500 and about 20,000 (ii) a diisocyanate, (iii) a branched, aliphatic C₈₋₃₆ primary alcohol, and (iv) optionally a modifier comprising two groups reactive with said diisocyante and a hydrophobic moiety. In one embodiment, the dispersion will comprise from about 10-60% by weight water, from about 10-40% by weight of the polyurethane polymer, and from about 10-70% by weight polyol. The polyol may be, without limitation, glycerin or a C₃₋₈ glycol such as propylene glycol, butylene pentylene glycol, hexylene glycol, caprylyl glycol, or the like. In one embodiment, the dispersion will comprise from about 10 to about 20% by weight water, from about 25 to about 35% by weight polyurethane polymer, and from about 45 to about 65% by weight propylene glycol. In another embodiment, the dispersion will comprise about 15% by weight water, about 30% by weight polyurethane polymer, and about 55% by weight propylene glycol. In yet another embodiment, the dispersion will comprise from about 30 to about 50% by weight water, from about 15 to about 20% by weight polymer, and from about 30 to about 50% by weight propylene glycol. In still another embodiment, the dispersion will comprise about 40% by weight water, about 20% by weight polymer, and about 40% by weight propylene glycol.

These and other aspects of the invention will be better understood by reference to the following detailed description of the invention.

### BRIEF DESCRIPTION OF THE FIGURES

FIG. 1 illustrates the near constant viscosity over varying shear rates for different compositions comprising a polyurethane polymer according to the invention. In the figure, “A” and “E” represent an aqueous system comprising, respectively, 4 and 5 weight % polyurethane polymer in water, “B” and “F” represent, respectively, a 4 and 5 weight % polyurethane polymer aqueous system, each further comprising 10 weight % Kobo GLW60GBAP, a black pigment dispersion. “C” represents a 4 weight % polyurethane polymer aqueous system further comprising 0.75 weight % Kobo GLW55GBAP, a red pigment dispersion. “D” represents an emulsion having an 80% aqueous phase and a 20% oil phase, where the oil phase comprises 90% Finsolve (C₁₂₋₁₅ Benzoate) and 10% POE (PEG 100 Stearate or PEG 6000 monostearate); and where the aqueous phase comprises the polyether polyurethane polymer from Rheolate® 288, such that the polymer comprises 4 weight % of the total emulsion composition.

FIG. 2 illustrates dramatic self-leveling properties of a mascara formulation comprising a polyurethane polymer according to the invention.

### DETAILED DESCRIPTION

It has surprisingly been found that cosmetic compositions comprising certain associative thickeners, including polyether polyurethane polymers, capable of forming cosmetic films exhibit properties of improved flow and self-leveling. The compositions are useful, in particular, for providing cosmetic compositions such as mascaras which provide films that self-level and substantially eliminate clumps and surface imperfections. The compositions of the present invention can include, comprise, consist essentially of or consist of the components of the present invention as well as other ingredients described herein. As used herein, “consisting essentially of” means that the composition or component may include additional ingredients, but only if the additional ingredients do not materially alter the basic and novel characteristics of the claimed compositions or methods.

The cosmetic compositions according to the invention have properties characteristic of Boger fluids, taking on the properties of the Boger fluid contained therein. A “Boger-like fluid” as used herein refers to any fluid that demonstrates one or more properties characteristic of Boger fluids, such as one or more of the unique rheological properties of such fluids. For example, a Boger fluid characteristically has a viscosity (η) that does not increase, or does not appreciably or significantly increase, with increasing shear (γ). Unless otherwise indicated, all compositions are on a weight basis, by weight of the total composition.

In some embodiments, the cosmetic compositions maintain a substantially constant viscosity over a wide range of shear rates, for example from about 0.01 sec⁻¹ to about 10 sec⁻¹. Thus, for example, some compositions according to the invention will exhibit a variance in viscosity of less than one order of magnitude over a range of shear rates spanning two, three or four orders of magnitude. By “substantially constant viscosity” is meant that the viscosity change is within the approximate range of ±50%, ±40%, ±30%, ±20%, or ±10% across a one order of magnitude change in shear rate, e.g., from about 0.01 to about 0.1 sec⁻¹, from about 0.1 to about 1 sec⁻¹ from about 1 to about 10 sec⁻¹, or from about 10 to about 100 sec⁻¹. In some embodiments, the cosmetic compositions are characterized by an increasing viscosity in response to increasing shear.

Cosmetic compositions of the instant invention show one or more properties characteristic of a Boger fluid, including, but not limited to, improved flow characteristics. The Boger-like flow characteristics can result in cosmetic formulations with unique aesthetics, texture, break, and/or renewable shine. For example, the inventive compositions exhibit an evening out of the product after application to a surface to produce a smoother finish, e.g., a finish with fewer imperfections, dumps, breaks or cracks, and/or an enhanced shine or gloss.

In some particularly preferred embodiments, the cosmetic compositions are self-leveling when applied to an integument (skin, nails, hair, lashes, etc.). These compositions exhibit a self-leveling property that is autonomous, requiring no intervention on the part of the user. Within a period of less than a few minutes (e.g., less than three, less than two, or less than one minute) after application to the integument, the resulting films are substantially smoother than comparable products which lack Boger fluid properties. Without wishing to be bound by any theory, it is believed that the compositions having Boger fluid properties exhibit greater autonomous flow after mechanical shear has ceased, for example, after being applied to a surface, as compared to conventional cosmetics and thus the inventive compositions will continue to correct surface imperfections and clumps for a substantial period of time after application, resulting in a smoother, glossier coating.

Broadly speaking, the invention embraces the use of any formulation that has Boger fluid characteristics, and in particular a constant or increasing viscosity over a range of shear rates. The formulations may include one or more polymers which impart the necessary rheological properties. Such polymers may be referred to as “self-leveling actives” of the compositions. The polymers may be natural, modified natural and/or synthetic polymers. Examples of synthetic polymers include polyurethanes, in particular polyether polyurethane polymers. Such polymers include those known as a “rheolate” or “associative thickener,” because the thickening arises as a result of hydrophobic associations between hydrophobic moieties in the rheolate molecules and other hydrophobic surfaces, such as on other particles or pigments in the system to be thickened.

In some preferred embodiments, the cosmetic compositions comprise an associative thickener, and in particular a polyurethane polymer. Polyurethane polymers are the polymerization products of a polyol component and a polyisocyante component. In some preferred embodiments, the polyurethane polymer results from the reaction of a mixture comprising (i) a polyether polyol component, (ii) a diisocyanate component, (iii) a branched, aliphatic alcohol for capping the polymer, and (iv) optionally a modifier comprising two reactive groups and a hydrophobic moiety. Other polyurethanes polymers are described in U.S. Pat. No. 5,973,063 to Doolan et al., herein incorporated by reference.

The polyol component is typically a linear or branched hydrocarbon comprising two or more reactive hydroxyl groups, typically primary hydroxyl groups. The polyol component will most often comprise diol molecules but may comprise some amount of more highly functionalized molecules to introduce cross-linking and branching into the polymer chain. Preferred polyols are polyether polyols.

The polyether polyol component (i) used in the present invention, can be selected from a variety of commercial products, and preferably has a representative formula for the repeating unit as follows:

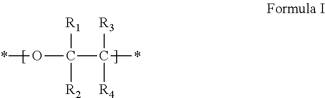

wherein R₁, R₂, R₃, and R₄ are each independently selected from the group consisting of H, CH₃ and C₂H₅. In some particularly preferred embodiments, the average molecular weight of the polyether polyol is from about 100 to about 50,000, more preferably from about 200 or about 500 to about 20,000, even more preferably from about 2,000 to about 14,000, and most preferably form about 2,000 to about 10,000 g/mol.

The polyether polyol typically have a hydroxyl moiety at each terminus, or substantially at each terminus, of the polyether chain and/or have one end of the polyether chain linked to a central molecule that itself has at least one additional active hydrogen moiety and/or polyether chain attached thereto. Exemplary preferred polyether polyols useful for the present invention include polyethylene glycol; random and block copolymers of ethylene glycol, copolymers with propylene glycol and/or butylene glycol; and polyether polyol adducts of glycerol, ethylene glycol with glycerol, trimethylolpropane, and pentaerythritol, and mixtures thereof. Particularly preferred polyether polyols are polyethylene glycol and mixtures of two polyethylene glycols of differing molecular weight.

The diisocyanate component (ii) used in the instant invention will contain two isocyanate groups per molecule and may be linear or branched, aliphatic, cycloaliphatic, and/or aromatic. Exemplary diisocyanates useful in the preparation of the associative thickener include 1,6-hexamethylene diisocyanate; 1,4-tetramethylene diisocyanate; a product designated Vestanat TMDI made by Huls America, Inc., which is a 40:60 (by weight) mix of 2,2,4-trimethyl hexamethylene-1,6-diisocyanate and 2,4,4-trimethylhexamethylene-1,6-diioscyanate; 1-isocyanato-3-isocyanatomethyl-3,5,5-trimethylcyclohexane; 1,10-decamethylene diisocyanate; 4-chloro-1,3-phenylene diisocyanate; 1,4-cyclohexylene diisocyanate; 4,4′-methylene bis-(isocyanatocyclohexane); m- and p-phenylene diisocyanate; 2,6- and 2,4-tolylene diisocyanate; xylene diisocyanate; 4,4′-biphenylene diisocyanate; 1,5-naphthylene diisocyanate; 4,4′-methylene diphenylisocyanate; 1,5-tetrahydronaphthylene diisocyanate; and mixtures thereof.

Those of skill in the art will appreciate that the diisocyanate molecule or molecules can contain functional moieties other than isocyanate if these additional moieties are substantially unreactive. Exemplary moieties, which do not adversely affect the reaction under conditions used to prepare the associative thickener, include: aldehyde; ketones; ethers; esters; halogenated hydrocarbons; amides; and tertiary amines.

The branched aliphatic alcohol (iii) will contain a hydroxyl moiety capable of reacting with the end group of the polyurethane polymer, and as such can act as a “capping agent.” The capping agent will cap the ends of the polymer chain, thereby terminating chain growth. The capping agent may contain other moieties, preferably where the other moieties are unreactive with an isocyanate functionality under the reaction conditions used in the preparation of the associative thickener. Such moieties include aldehydes, ketones, ethers, esters, hydrogenated hydrocarbons, tertiary amines, and amides.

A preferred set of branched alcohols useful as capping agents are represented by the following formula:

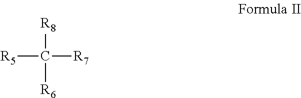

where R₅ is HO—CH₂—(CH₂)ₖ with k=0 to 4; R₆ is (CH₂)ₘCH₃ where m is 4 to 14; R₇ is (CH₂)ₙCH₃ where n is 2 to 10; and R₃ is H, CH₃, or C₂H₅. Preferred branched alcohols useful as capping agents include lateral branched chains. More preferably, the alcohol is a primary alcohol, and the hydroxyl moiety occurs on the end of a carbon chain of from about 3 to about 50 carbon atoms in length, more preferably from about 5 to about 40 carbon atoms in length, and most preferably from about 8 to about 36 carbon atoms in length.

The optional modifier (iv) will comprise at least one hydrophobic moiety and at least two groups reactive with the diisocyanate. Exemplary active hydrogen moieties include primary amino, secondary amino, hydroxy, carboxy and mercapto groups. The optional modifier is used for adjusting the hydrophobic character of the other components, so that a suitable Boger-fluid polymer can be obtained. The optional modifier preferably has at least about 10 carbon atoms, with the hydrophobic group having no moieties reactive with the diisocyanate or polyether polyol components. The hydrophobic group is typically a hydrocarbon group, but it can contain moieties, such as halogen or alkoxy groups.

In some particularly preferred embodiments, a cosmetic composition comprising an associative thickener is provided, where the associative thickener comprises a polyether urea polyurethane polymer. The polymer can comprise repeating urea units within the generalized polyurethane polymer structure. Urea is represented as having the structure shown in Formula III below:

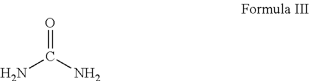

The polyurethane polymers of the instant invention can be obtained by polymerizing the employed components by any method known in the art such as by solution, suspension, or solvent-free or melt polymerization on a batch, continuous, or semi-continuous basis. The amounts of the various components will be selected as known in the art, e.g., as described in U.S. Pat. No. 5,973,063 to Doolan, et al., herein incorporated by reference. In a (preferred embodiment, the polymerization process provides the polyurethane polymer in the product sold under the trade name Rheolate® 288, which is used in the paint industry. As mentioned, Rheolate® 288 also is commercially available, e.g., from Elementis Specialties, Inc, New Jersey, and is supplied as a suspension in butyl carbitol and water. Cosmetic compositions of the instant invention comprising the polyurethane polymer do not contain butyl carbitol, which poses toxicity issues.

Rather, the polyurethane polymer is provided in an aqueous system, such as a water and/or propylene glycol system. The polymer may be dispersed, suspended or otherwise contained in an aqueous system suitable for cosmetic application. The aqueous system may also comprise any water soluble/dispersible solvents suitable for forming a Boger fluid with the polymer. In certain preferred embodiments, the aqueous system comprises propylene glycol and/or water. For example, the cosmetic composition may comprise the polyether polymer of Rheolate® 288 but provided in combination with a water/polyol system such that the polymer/solvent system comprises from about 10-60% by weight water, from about 10-40% by weight of the polyurethane polymer, and from about 10-70% by weight polyol, and in particular about 15 to 40% by weight water, about 20-30% by weight polymer, and about 40-55% polyol. The polyol may be, without limitation, glycerin or one or more C₃₋₈ glycols such as propylene glycol, butylene glycol, pentylene glycol, hexylene glycol, caprylyl glycol, or the like. Propylene glycol is preferred. In one embodiment, the polymer system will comprise about 15% by weight water, about 30% by weight polyurethane polymer, and about 55% by weight propylene glycol. In another embodiment, the dispersion will comprise about 40% by weight water, about 20% by weight polyurethane polymer, and about 40% by weight propylene glycol. Such suspensions, for example, retain Boger fluid-like properties and avoid the toxicity issues of the butyl carbitol suspension. See Example 1 below. Other Boger fluid polymers can be used instead of or in combination with the polyurethane polymer in the same or other systems. For example, other Boger fluid polymers can be provided in an aqueous system suitable for use in cosmetics.

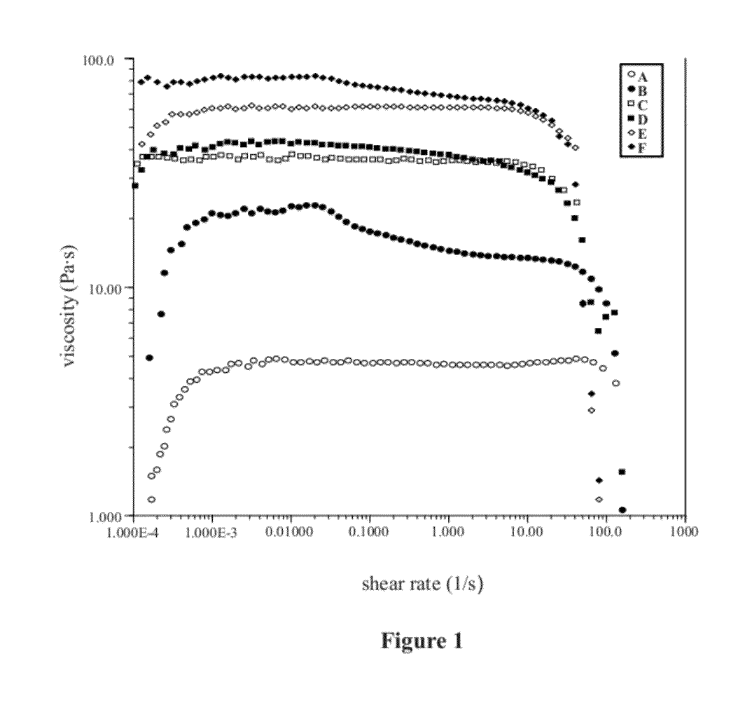

In some embodiments, the viscosity of the cosmetic composition, e.g., a composition comprising a polyurethane polymer in an aqueous system, is in the range of about 1 to about 100 Pa·s⁻¹, for example where the viscosity is measured at 25° C., and over a range of shear rates, from about 10⁻⁴ to about 100 s⁻¹. In preferred embodiments, the viscosity is at least about 10, at least about 20, at least about 30, at least about 40, at least about 50, at least about 80, or at least about 90 Pa·s⁻¹, for example, measured at 25° C., and over a range of shear rates, from about 10⁻⁴ to about 100 s⁻¹. See Example 1 below, FIG. 1. The viscosity of Boger fluids can be measured as known in the art. For example, a Rheomat RM 180 viscometer can by used, fitted with a No. 4 rotor, with the measurements being carried out after spinning the rotor for about 10 minutes (to allow stabilization of the viscosity and of the rotor spin speed). See, e.g., U.S. Pat. No. 6,375,941. Typically, measurements are made to room temperature (about 25° C.) and over a range of shear rates.

Cosmetic compositions of the instant invention generally comprise an amount of a Boger fluid polymer effective to impart one or more Boger fluid-like properties to the compositions. In some embodiments, the polymer is in an amount from about 0.01 to about 30 weight %, based on the total weight of the composition. In some embodiments, the polymer is in an amount from about 0.1 weight % to about 25 weight %, from about 0.5 weight % to about 20 weight %, from about 1 weight % to about 15 weight %, or about 2 to about 10 weight %, based on the total weight of the composition. In certain preferred embodiments, the polymer is a polyurethane polymer, such as polyether urea polyurethane.

The cosmetic compositions of the invention will typically contain from about 10-80%, more typically from 25-70%, and in particular from about 35-60% by weight water. In various embodiments the compositions comprise 60%, or 55%, or 50%, or 45%, or 40% by weight water.

The cosmetic compositions of the invention often will contain a polyol, generally in an amount from about 0.5-40%, more typically from 1-25%, in particular from about 2-15% by weight polyol, and especially propylene glycol in an amount of from about 2-10% by weight in various embodiments the compositions comprise 30%, or 20%, or 15%, or 10%, or 5%, or 2.5% by weight polyol.

In some embodiments, the cosmetic composition further comprises one or more of the components selected from Kobo GLW60GBAP; Kobo GLW55GBAP; Finsolve (C₁₂₋₁₅ Benzoate) and POE (PEG 100 Stearate or PEG 6000 monostearate). These additional components in certain amounts have been shown to not affect, or to not substantially and/or significantly affect, the Boger-like properties of some compositional embodiments of the instant invention. See Example 1, FIG. 1. Kobo GLW60GBAP is a black pigment dispersion, comprising iron oxides, water, glycerin, ammonium polyacrylate, and methyparaben. An aqueous system comprising about 3 to about 5 weight % of the polyether polymer from Rheolate® 288 retains Boger-like properties with the addition of about 0.75 to about 10 weight % Kobo GLW60GBAP. Kobo GLW55GBAP is a red pigment dispersion, comprising iron oxides, water, glycerin, ammonium polyacrylate, and methyparaben. An aqueous system comprising about 3 to about 5 weight % of the polyether polymer from Rheolate® 288 retains Boger-like properties with the addition of about 0.75 to about 10 weight % Kobo GLW55GBAP.

Finsolve (C₁₂₋₁₅ Benzoate) and POE (PEG 100 Stearate or PEG 6000 monostearate) can be used to provide emulsions with the polyurethane polymer in water that also retain Boger-like properties. In some embodiments, the emulsion composition comprises from about 20 to about 25% of an oil phase composed of 90% Finsolve (C₁₂₋₁₅ Benzoate) and 10% POE; where the remaining about 80 to about 75% of the composition comprises an aqueous phase comprising the polyether polyurethane polymer from Rheolate® 288, such that the polymer comprises about 4 weight % of the total emulsion composition. See again Example 1, FIG. 1.

Cosmetic Use of Boger Fluid Compositions

Another aspect of the instant invention relates to cosmetic use of compositions comprising certain Boger fluids. Cosmetic compositions having Boger-like properties afford surprising and unique characteristics, desirable in cosmetic products. Such properties include flow characteristics, renewable shine and gloss, and the ability to self-level, which can translate to cosmetic products that provide desirable aesthetics, sensory and/or practical qualities.

In some embodiments, the cosmetic composition comprising a Boger fluid provides for ease of application, in particular where the consumer desires achieving a smooth, even finish. Boger-like flow characteristics can make it easier to achieve a smooth and/or even finish upon application of the cosmetic. For example, a consumer may desire a smooth and/or even application of a foundation, a lip product, a eye product, or other topical composition. A “topical composition” refers to a composition for use on an outer surface or integument of the body, e.g., on the skin, e.g., on the skin of the face, lips, neck, hands, arms, stomach, back, legs, and the like; or for coating the surface of a keratin fiber, such as the hair, eyelashes, eyebrows, and the like. Mascara products, for example, are particularly intended for essentially longilinear keratin fibers, such as the eyelashes, eyebrows, and hair, including false eyelashes and wigs. The eyelash has two distinct structures—the hair follicle, which resides in the skin; and the hair shaft or hair fiber, visible above the skin. Mascara products described herein are intended for coating the shaft of a keratin fiber, such as the shaft of an eyelash, by forming a film thereupon.

In certain preferred embodiments, the cosmetic composition comprising a Boger fluid has a self-leveling property. As discussed, self-leveling can allow for autonomous flow of a topical composition after its application to a surface, thus easing application and achievement of a smooth, uniform, and/or even finish. For example, a self-leveling or self-correcting mascara can ease application and allow for achieving a uniform look, where lashes are uniformly and/or evenly coated with the cosmetic composition. In the absence of Boger fluid-like mascara products tend to clump on the eyelashes resulting in a non-uniform look, for example due to the mascara's relatively high wax content and/or inability to flow. Such products often require the consumer to repeatedly brush or comb through the lashes, during and/or after application, in an effort to remove clumps.

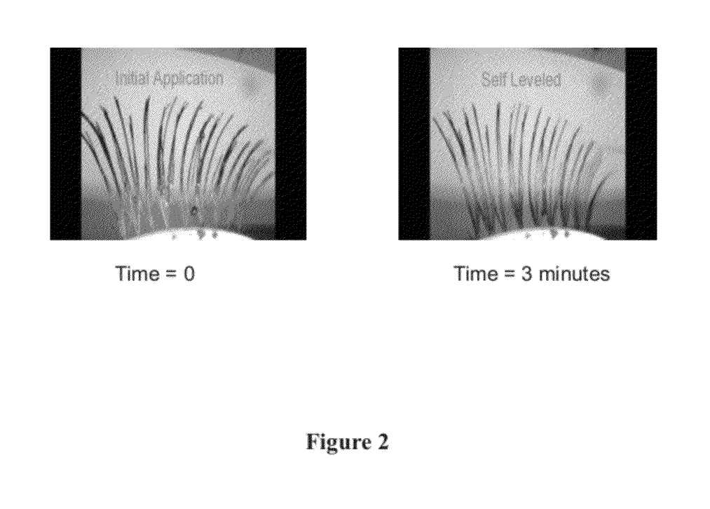

Cosmetic compositions described herein having Boger fluid-like properties, including a self-leveling property, can find use in improved mascara products, such as an anti-clump mascara. The anti-clump mascara preferably allows ease of application and achievement of a smooth, clump-free look. For example, in some embodiments, the composition is easy to apply to the length of the eyelash and any clumps and surface irregularities that occur upon initial application smooth over the lash with time, or smooth out to a great extent than that observed with mascaras that lack a composition described herein and that do not go smooth over time. In certain preferred embodiments, for example, initially existing clumps and surface irregularities will be substantially eliminated within a few minutes or less, e.g., less than about 3 minutes, less than about 2 minutes, or less than about one minute, without requiring action by the user. Self-leveling of an anti-clump mascara of some embodiments of the instant invention is illustrated in Example 2 and FIG. 2 below. Unique flow properties can also aid decoupling of the mascara formulation from the mascara wand or brush, e.g., so that the formulation comes off the brush or wand easily, preferably allowing significant “pay-off” of the formulation onto the lashes. Mascara compositions according to the instant invention thus can provide a product which is fast and easy to apply, coats the eyelashes well, and provides a make-up effect that is natural and/or comfortable. The mascara product can be a make-up product, a make-up base, or a topcoat to be applied over make-up.

In some particularly preferred embodiments, a method for applying a smooth cosmetic film to eyelashes is provided. The method may comprise forming an initial coating on the eyelashes with a composition described herein, such as a composition comprising an associative thickener capable of forming a film on the shaft of a keratin fiber. The coating can then be allowed to self-level, preferably to provide a coating having reduced clumping and/or a smoother and/or more even surface as compared to the initial coating. For example, clumps and/or surface irregularities preferably are substantially eliminated within about five minutes, within about three minutes, or within about a minute or less after initial application of the mascara. Such methods take advantage of the autonomous flow and/or self-correcting nature of cosmetic compositions described herein, to provide surprisingly effective results with respect to applying mascara more smoothly and evenly to eyelashes, with little effort on the part of the user.

In some embodiments, the cosmetic composition comprising a Boger fluid, such as an associative thickener herein described, provides for longer wear, in particular where the consumer desires maintaining a smooth, even finish for an extended period of time. Boger-like flow characteristics can allow the already-applied cosmetic composition to re-distribute over time and/or with slight movements, e.g., with movements of the facial or lip muscles, or with the blinking of the eyelids. This can reduce or prevent breaks or cracks from forming on the surface to which the composition was applied, with the result that a smooth and/or even appearance is maintained for an extended period of time. For example, a consumer may desire a smooth and/or even appearance for an extended period of time of a foundation, rouge, powder, lip product (colored or gloss), eye product, including a mascara, or other topical composition. The time period may be a few hours, several hours, a day, or even two days in some embodiments.

In some particularly preferred embodiments, the cosmetic composition has a self-leveling property that facilitates re-distribution and maintenance a smooth and/or even appearance or a uniform coat. This autonomous redistribution can help maintain a more natural and/or fresher look. For example, a self-correcting mascara product can re-distribute over time to maintain a uniform coat over the eyelashes, preferably over the entire or almost the entire length of the shaft of the eyelash keratin fiber, reducing clumping and/or keeping eyelashes from sticking together over a period of wear, for example, a few hours, several hours, a day, or even two days in some embodiments.

In some embodiments, the cosmetic composition comprising a Boger fluid, such as an associative thickener herein described, provides for a shiny and/or glossy finish. Boger fluid-like characteristics can create a shiny and/or glossy appearance, for example, where the composition flows to form a smooth and/or even layer over the skin or hair that better reflects incident light. This can result in the appearance of a shine or gloss on the surface to which the composition was applied. For example, a consumer may desire a shiny and/or glossy appearance of the face, lips, eyelashes, or hair, and the cosmetic compositions described herein can find use in topical compositions for application to such surfaces.

In certain preferred embodiments, a composition comprising a Boger fluid, such as an associative thickener herein described, is used in a lip product, such as a lip stick or lip gloss, to provide a shiny appearance to the lips. In some embodiments, a composition comprising a Boger fluid, such as an associative thickener herein described, is used in personal care products for the skin, such as a body lotion, body wash or body spray, where the composition can impart the appearance of a healthy shine to the skin. In certain preferred embodiments, a composition comprising a Boger fluid, such as an associative thickener herein described, is used in a lip product, such as a lip stick or lip gloss, to provide a shiny appearance to the lips. In some embodiments, a composition comprising a Boger fluid, such as an associative thickener herein described, is used in personal care products for the hair, such as a shampoo, conditioner or mousse, where the composition can impart the appearance of a healthy shine to the hair.

In some embodiments, the cosmetic composition for providing a shiny and/or glossy look also permits extended wear. As noted above, Boger-like flow characteristics can allow for the already-applied cosmetic composition to re-distribute over time and/or with slight movements, e.g., with movements of the facial or lip muscles or with the blinking of the eyelids, reducing and/or preventing clumping, breaks or cracks. This can result in the maintenance of a smooth and/or even appearance for an extended period of time, and consequently for the maintenance or apparent renewal of a high shine and/or gloss. For example, a consumer may desire a shiny and/or glossy appearance for an extended period of time from a foundation, lip product, eye product, including a mascara, or other topical composition. The time period may be a few hours, several hours, a day, or even two days in some embodiments. In certain preferred embodiments, a composition comprising a Boger fluid, such as an associative thickener herein described, is used in a long wear and/or renewable tip product, such as a tip stick or tip gloss, to provide a shiny appearance to the lips for an extended period of time.

In certain particularly preferred embodiments, the cosmetic composition comprising a Boger fluid exhibits a self-leveling property that aids in creating, renewing, and/or maintaining a shiny or glossy appearance. Self-leveling can allow autonomous re-distribution of the cosmetic composition to create, re-create and/or keep a smooth and/or even finish that better reflects incident light. Such compositions can find use, for example, as a renewable and/or long wear tip gloss, lipstick or other tip product, or as an eye product, such as a renewable and/or long wear anti-clump mascara.

In some embodiments, the cosmetic composition comprising a Boger fluid, such as an associative thickener herein described, provides a certain sensory or tactile experience. Boger fluid-like characteristics can create different kinds of texture, not found in cosmetic products lacking Boger fluids. The unique texture can translate to a silky, smooth, or gliding sensation during and/or after application of the composition to the skin.

The cosmetic compositions described herein find use in improved cosmetic products, including for example, easy-to-apply foundation, easy-to-apply anti-clump mascara, and renewable or tong-wear foundation, mascara, lipstick and lip gloss. In preferred embodiments, the cosmetic compositions comprise a Boger fluid polymer, such as an associative thickener herein described. For example, a cosmetic composition can comprise an amount of polyurethane polymer effective to impart the desired qualities to the cosmetic. In certain preferred embodiments, the polymer is a polyether polyurethane polymer or a polyether urea polyurethane polymer, more preferably in an amount effective to impart self-leveling properties to the cosmetic composition, where the composition is to be topically applied to skin or hair, including the eyelashes.

Cosmetic Formulations Using Boger Fluids

The compositions according to the instant invention can be formulated in a variety of forms for topical application. The composition may be formulated in a variety of product forms suitable for application to the skin, hair, eyelashes, or eyebrows, such as, for example, a lotion, cream, serum, spray, aerosol, ointment, essence, gel, paste, patch, pomade, solution, towelette, mask, foam, elixir, concentrate, or any other liquid or semisolid form.

Suitable forms may depend on the type of cosmetic product. For example, for a foundation, the composition is preferably formulated as a lotion, cream, liquid, or mousse; for an eye eyeliner, the composition is preferably formulated as a liquid; for an eye shadow, the composition is preferably formulated as a cream; for a lip product, the composition is preferably formulated as a paste or cream; for a mascara product, the composition is preferably formulated as a paste or cream, preferably supplied in a reservoir with an applicator, wand, or brush integral with a removable closure.

The compositions will comprise an effective amount of a Boger fluid, by which is meant an amount sufficient to impart one or more Boger fluid-like properties to the formulated product. An “amount effective” or an “effective amount” to impart a particular property refers to the amount of Boger fluid, e.g., the amount of polyurethane polymer, required to produce at least one measurable Boger-like property. For example, the Boger fluid polymer may be present in an amount from about 0.01 weight % to about 30 weight %, from about 0.1 weight % to about 25 weight %, from about 0.5 weight % to about 20 weight %, from about it weight % to about 15 weight %, or about 2 to about 10 weight %, based on the total weight of the composition.

In certain particularly preferred embodiments, the cosmetic composition is an anti-clump mascara comprising an effective amount of polyurethane polymer to facilitate self-leveling and reduce clumping. In some preferred embodiments, polyurethane polymer is present is in an amount from about 0.01 weight % to about 30 weight %, from about 0.1 weight % to about 25 weight %, from about 0.5 weight % to about 20 weight %, from about 1 weight % to about 15 weight %, or about 2 to about 10 weight %, based on the total weight of the composition.

The compositions can include a cosmetically acceptable vehicle. Such vehicles may take the form of any known in the art suitable for application to skin or hair and may include water (e.g., deionized water); vegetable oils; mineral oils; esters such as octal palmitate, isopropyl myristate and isopropyl palmitate; ethers such as dicapryl ether and dimethyl isosorbide; isoparaffins such as isooctane, isododecane and isohexadecane; silicone oils such as cyclomethicone, dimethicone, dimethicone cross-polymer, polysiloxanes, and their derivatives, preferably organomodified derivatives; hydrocarbon oils such as mineral oil, petrolatum, isoeicosane, and polyisobutene; polyols such as propylene glycol, glycerin, butylene pentylene glycol, and hexylene glycol; waxes such as beeswax and botanical waxes; or any combinations or mixtures of the foregoing.

The vehicle may comprise an aqueous, polyol or hydropolyol phase, an oil phase, a silicone phase, and compatibly suitable combinations thereof, with the aqueous, polyol or aqueous/polyol phase typically containing the Boger fluid polymer of the invention dissolved, dispersed or suspended therein. The cosmetically acceptable vehicle may comprise an aqueous, polyol, or hydropolyol gel composition, or the cosmetically acceptable vehicle may also comprise an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like, for example, having the appearance of a cream, gel or micro-emulsions. The emulsion may include an emulsifier, such as a nonionic, anionic or amphoteric surfactant. Oil-in-water emulsions are preferred.

The aqueous phase of the emulsion may include water, one or more additional water soluble solvents such as polyols, and one or more water soluble or water dispersible active components. The aqueous phase of the emulsion also typically contains the Boger fluid polymer, which is suspended or dispersed therein. The cosmetically acceptable vehicle can comprise component(s) compatible with the system used. For example, polyols, preferably propylene glycol, can form a polymer suspension or dispersion as hereinbefore described, in combination with or without water, which suspension/dispersion is subsequently incorporated into the cosmetic composition. In certain particular embodiments, the formulation comprises a mascara, where the mascara formulation comprises a water/propylene glycol aqueous phase that further contains the self-leveling polyether polymer or the preformed suspension/dispersion thereof.

In some embodiments, the mascara formulation does not comprise an oil or an oil phase. In some embodiments, the mascara formulation does not comprise an emulsion. Formulations corresponding to other types of cosmetics, for example, foundations or lip products, may also lack an oil phase and/or an emulsion, in other embodiments, the mascara or other cosmetic formulation may contain an oil phase, wax, and/or an emulsion.

The oil phase of the emulsion preferably has one or more organic compounds, including emollients; humectants (such as butylene glycol, propylene glycol, Methyl gluceth-20, and glycerin); other water-dispersible or water-soluble components including thickeners such as Veegum or hydroxyalkyl cellulose; gelling agents, such as high MW polyacrylic acid, i.e. CARBOPOL 934; and mixtures thereof. The emulsion may have one or more emulsifiers capable of emulsifying the various components present in the composition.

Compounds suitable for use in the oil phase include without limitation, vegetable oils; esters such as octyl palmitate, isopropyl myristate and isopropyl palmitate; ethers such as dicapryl ether; isoparaffins such as isooctane, isododecane and isohexadecane; silicone oils such as dimethicones, cyclic silicones, and polysiloxanes; hydrocarbon oils such as mineral oil, petrolatum, isoeicosane and polyisobutene; natural or synthetic waxes; one or more oil soluble active components, and the like, individually or in compatible combination. Suitable hydrophobic hydrocarbon oils may be saturated or unsaturated, have an aliphatic character and be straight or branched chained or contain alicyclic or aromatic rings. The oil-containing phase may be composed of a singular oil or mixtures of different oils.

Hydrocarbon oils include those having 6-20 carbon atoms, more preferably 10-16 carbon atoms. Representative hydrocarbons include decane, dodecane, tetradecane, tridecane, and C₈₋₂₀ isoparaffins. Paraffinic hydrocarbons are available from Exxon under the ISOPARS trademark, and from the Permethyl Corporation. In addition, C₈₋₂₀ paraffinic hydrocarbons such as C₁₂ isoparaffin (isododecane) manufactured by the Permethyl Corporation having the tradename Permethyl 99ATM are also contemplated to be suitable. Various commercially available C₁₆ isoparaffins, such as isohexadecane (having the tradename Permethyl RTM) are also suitable. Examples of preferred volatile hydrocarbons include polydecanes such as isododecane and isodecane, including for example, Permethyl-99A (Presperse Inc.) and the C₇-C₈ through C₁₂-C₁₅ isoparaffins such as the Isopar Series available from Exxon Chemicals. A representative hydrocarbon solvent is isododecane.

The oil phase may comprise one or more waxes, including for example, rice bran wax, carnauba wax, ouricurry wax, candelilla wax, montan waxes, sugar cane waxes, ozokerite, shellac wax, rice bran wax, polyethylene waxes, Fischer-Tropsch waxes, beeswax, botanical waxes, microcrystalline wax, silicone waxes, fluorinated waxes, paraffin wax, synthetic waxes, and any combination thereof. “Wax” or “waxes”, as used herein, generally refers to compounds that are solid at room temperature (about 25° C.), and having a melting point ranging from about 45° C. to about 110° C. The wax component may be incorporated into the compositions of the invention in an amount of up to about 25% by weight, typically from 0 to about 20 weight %, from about 0.5 to about 15 weight %, and from about 1 to about 12 weight %. Suitably, the compositions can contain 2, 4, 6, 8, 10, or 12 weight % wax. For example, in some particularly preferred embodiments, a cosmetic composition is provided that comprises from about 2 to about 12 weight % of waxes and about 4 to about 6 weight % of the polymer of Rheolate® 288 in a water/propylene glycol aqueous system, where the polymer/aqueous system itself comprises about 20 weight % polymer; about 40 weight % water; and about 40 weight % propylene glycol. In some other particularly preferred embodiments, a cosmetic composition is provided that comprises from about 0 to about 24% waxes and from about 2 to about 10 weight % of the polymer of Rheolate® 288 in a water/propylene glycol aqueous system, where the polymer/aqueous system itself comprises about 20 weight % polymer; about 40 weight % water; and about 40 weight % propylene glycol. In some even more preferred embodiments, a cosmetic composition is provided that comprises from about 1.5 to about 12 weight % waxes and about 5 weight % of the polymer of Rheolate® 288 in a water/propylene glycol aqueous system, where the polymer/aqueous system itself comprises about 20 weight % polymer; about 40 weight % water; and about 40 weight % propylene glycol. See Example 3 below. In some other even more preferred embodiments, a cosmetic composition is provided that comprises about 12 weight % wax and about 2.5 weight % of the polymer of Rheolate® 288 in a water/propylene glycol aqueous system, where the polymer/aqueous system itself comprises about 30 weight % polymer; about 15 weight % water; and about 55 weight % propylene glycol. See Example 4 below.

The oil phase may comprise one or more volatile and/or non-volatile silicone oils. Volatile silicones include cyclic and linear volatile dimethylsiloxane silicones. In some embodiments, the volatile silicones may include cyclodimethicones, including tetramer (D4), pentamer (D5), and hexamer (D6) cyclomethicones, or mixtures thereof. Particular mention may be made of the volatile cyclomethicone-hexamethyl cyclotrisiloxane, octamethyl-cyclotetrasiloxane, and decamethyl-cyclopentasiloxane. Suitable dimethicones are available from Dow Corning under the name Dow Corning 200® Fluid and have viscosities ranging from 0.65 to 600,000 centistokes or higher. Suitable non-polar, volatile liquid silicone oils are disclosed in U.S. Pat. No. 4,781,917, herein incorporated by reference in its entirety. Additional volatile silicones materials are described in Todd et al., “Volatile Silicone Fluids for Cosmetics”, Cosmetics and Toiletries, 91:27-32 (1976), herein incorporated by reference in its entirety. Linear volatile silicones generally have a viscosity of less than about 5 centistokes at 25° C., whereas the cyclic silicones have viscosities of less than about 10 centistokes at 25° C. Examples of volatile silicones of varying viscosities include Dow Corning 200, Dow Corning 244, Dow Corning 245, Dow Corning 344, and Dow Corning 345, (Dow Corning Corp.); SF-1204 and SF-1202 Silicone Fluids (G.E. Silicones), GE 7207 and 7158 (General Electric Co.); and SWS-03314 (SWS Silicones Corp). Linear, volatile silicones include low molecular weight polydimethylsiloxane compounds such as hexamethyldisiloxane, octamethyltrisiloxane, decamethyltetrasiloxane, and dodecamethylpentasiloxane, to name a few.

Non-volatile silicone oils will typically comprise polyalkylsiloxanes, polyarylsiloxanes, polyalkylarylsiloxanes, or mixtures thereof. Polydimethylsiloxanes are (preferred non-volatile silicone oils. The non-volatile silicone oils will typically have a viscosity from about 10 to about 60,000 centistokes at 25° C., preferably between about 10 and about 10,000 centistokes, and more preferred still between about 10 and about 500 centistokes; and a boiling point greater than 250° C. at atmospheric pressure. Non limiting examples include dimethyl polysiloxane (dimethicone), phenyl trimethicone, and diphenyldimethicone. The volatile and non-volatile silicone oils may optionally be substituted will various functional groups such as alkyl, aryl, amine groups, vinyl, hydroxyl, haloalkyl groups, alkylaryl groups, and acrylate groups, to name a few. Based on the teachings herein, a person skilled in the art will be able to select any of these silicone oils or other optional additives, and/or the amount thereof, such that the desirable properties of the cosmetic compositions described herein can be conserved.

In some embodiments, emulsions comprising Finsolve (C₁₂₋₁₅ Benzoate) and/or POE (PEG 100 Stearate or PEG 6000 monostearate) can be used. For example, in some embodiments, as discussed above, the emulsion composition comprises from about 20 to about 25% of an oil phase composed of 90% Finsolve (C₁₂₋₁₅ Benzoate) and 10% POE (PEG 100 Stearate or PEG 6000 monostearate); where the remaining about 80 to about 75% of the composition comprises an aqueous phase comprising the polyether polyurethane polymer from Rheolate® 288, such that the polymer makes up about 4% by weight of the total emulsion composition. Such emulsions have been shown to retain Boger-like properties of the polyurethane polymer. See again Example 1, FIG. 1. Based on these teachings, one of skill of the art will be able to select other oil phase components, in appropriate amounts, to provide additional emulsion formulations comprising polyether polyurethane polymer and having Boger-like properties, such as self-leveling.

Non-limiting emulsifiers include emulsifying waxes, polyether polyols, polyethers, mono- or di-ester of polyols, ethylene glycol mono-stearates, glycerin mono-stearates, glycerin di-stearates, silicone-containing emulsifiers, soya sterols, acrylates, fatty acids such as stearic acid, fatty acid salts, and mixtures thereof. The preferred emulsifiers include soya sterol, stearic acid, emulsifying wax, acrylates, silicone containing emulsifiers and mixtures thereof. Other specific emulsifiers that can be used in the composition of the present invention include, but are not limited to, one or more of the following: C₁₀₋₃₀ alkyl acrylate crosspolymer; Dimethicone PEG-7 isostearate, acrylamide copolymer; mineral oil; sorbitan esters; polyglyceryl-3-diisostearate; sorbitan monostearate, sorbitan tristearate, sorbitan sesquioleate, sorbitan monooleate; glycerol esters such as glycerol monostearate and glycerol monooleate; polyoxyethylene ethers such as polyoxyethylene cetyl ether and polyoxyethylene stearyl ether; polyoxyethylene glycol esters; polyoxyethylene sorbitan esters; dimethicone copolyols; polyglyceryl esters such as polyglyceryl-3-diisostearate; glyceryl laurate; Steareth-2, Steareth-10, and Steareth-20, to name a few. Additional emulsifiers are provided in the INCI Ingredient Dictionary and Handbook 11th Edition 2006, the disclosure of which is hereby incorporated by reference.

These emulsifiers typically will be present in the composition in an amount from about 0.001% to about 10% by weight, in particular in an amount from about 0.01% to about 5% by weight, and more preferably, from about 0.1% to about 3% by weight.

The water-in-silicone emulsion may be emulsified with a nonionic surfactant (emulsifier) such as, for example, polydiorganosiloxane-polyoxyalkylene block copolymers, including those described in U.S. Pat. No. 4,122,029, the disclosure of which is hereby incorporated by reference. These emulsifiers generally comprise a polydiorganosiloxane backbone, typically polydimethylsiloxane, having side chains comprising -(EO)ₘ— and/or —(PO)ₙ— groups, where EO is ethyleneoxy and PO is 1,2-propyleneoxy, the side chains being typically capped or terminated with hydrogen or lower alkyl groups (e.g., C₁₋₆, typically C₁₋₃). Other suitable water-in-silicone emulsifiers are disclosed in U.S. Pat. No. 6,685,952, the disclosure of which is hereby incorporated by reference herein. Commercially available water-in-silicone emulsifiers include those available from Dow Corning under the trade designations 3225C and 5225C FORMULATION AID; SILICONE SF-1528 available from General Electric; ABU, EM 90 and EM 97, available from Goldschmidt Chemical Corporation (Hopewell, Va.); and the SILWET series of emulsifiers sold by OSI Specialties (Danbury, Conn.).

Examples of water-in-silicone emulsifiers include, but are not limited to, dimethicone PEG 10/15 crosspolymer, dimethicone copolyol, cetyl dimethicone copolyol. PEG-15 lauryl dimethicone crosspolymer, laurylmethicone crosspolymer, cyclomethicone and dimethicone copolyol, dimethicone copolyol (and) caprylic/capric triglycerides, polyglyceryl-4 isostearate (and) cetyl dimethicone copolyol (and) hexyl laurate, and dimethicone copolyol (and) cyclopentasiloxane. Preferred examples of water-in-silicone emulsifiers include, without limitation, PEG/PPG-18/18 dimethicone (trade name 5225C, Dow Corning), PEG/PPG-19/19 dimethicone (trade name BY25-337, Dow Corning), Cetyl PEG/PPG-10/1 dimethicone (trade name Abil EM-90, Goldschmidt Chemical Corporation), PEG-12 dimethicone (trade name SF 1288, General Electric), lauryl PEG/PPG-18/18 methicone (trade name 5200 FORMULATION AID, Dow Corning), PEG-12 dimethicone crosspolymer (trade name 9010 and 9011 silicone elastomer blend, Dow Corning), PEG-10 dimethicone crosspolymer (trade name KSG-20, Shin-Etsu), dimethicone PEG-10/15 crosspolymer (trade name KSG-210, Shin-Etsu), and dimethicone PEG-7 isostearate.

The water-in-silicone emulsifiers typically will be present in the composition in an amount from about 0.001% to about 10% by weight, in particular in an amount from about 0.01% to about 5% by weight, and more preferably, below 1% by weight. A person of skill in the art, based on the teachings herein, will be able to select any of these emulsifiers or other optional additives, and/or the amount thereof, such that the desirable properties of the cosmetic compositions described herein can be conserved.

The oil-containing phase of emulsions useful herein will typically comprise from about 1% to about 75%, preferably from about 5% to about 50%, and more preferably from about 20% to about 25% by weight, based on the total weight of the emulsion; and the aqueous phase will typically comprise from about 25% to about 99%, preferably from about 50% to about 95%, and more preferably from about 75% to about 80% by weight of the total emulsion. The aqueous phase will typically comprise from about 25% to about 100%, more typically from about 50% to about 95%, or often from about 40% to about 80% by weight water by weight water.

In some preferred embodiments, the cosmetic composition is a self-leveling mascara comprising a polyurethane polymer, more preferably polyether urea polyurethane polymer, in combination with one or more components commonly found in cosmetic formulations, including, for example, film formers, gums, preservatives, thickeners, pigment dispersions, waxes and the like. Such additional components can retard, augment, and/or have no effect on the self-leveling action of the mascara product. For example, the presence of alcohols can disrupt the viscosity of the system, reducing the ability of the composition to self-level, whereas the addition of about 0.1 to about 2 weight % of gums, more preferably about 0.1 to about 1 weight %, can increase the overall viscosity of the composition without disrupting, or without appreciably or significantly disrupting, the self-leveling properties. The gum may comprise but is not limited to xantham gum, sodium CMC, stearic acid, and/or triethanolamine. For example, in some embodiments, a cosmetic composition is provided that comprises about 0.1 to about 2 weight % of gum and about 2 to about 6 weight % of the polymer of Rheolate® 288 in a water/propylene glycol aqueous system, where the polymer/aqueous system itself comprises about 20 weight % polymer; about 40 weight % water; and about 40 weight % propylene glycol. In some embodiments, a cosmetic composition is provided that comprises about 0.1 to about 2 weight % of gum and about 2 to about 3 weight % of the polymer of Rheolate® 288 in a water/propylene glycol aqueous system, where the polymer/aqueous system itself comprises about 30 weight % polymer; about 15 weight % water; and about 55 weight % propylene glycol.

In some embodiments, the cosmetic composition comprises film formers. Film formers include, but are not limited to, polyurethanes and (meth)acrylate copolymers. For example, in some embodiments, a cosmetic composition is provided that comprises about it to about 20 weight % of film formers and about 2 to about 6 weight % of the polymer of Rheolate® 288 in a water/propylene glycol aqueous system, where the polymer/aqueous system itself comprises about 20 weight % polymer; about 40 weight % water; and about 40 weight % propylene glycol. In some embodiments, a cosmetic composition is provided that comprises about 1 to about 20 weight % of film formers and about 2 to about 3 weight % of the polymer of Rheolate® 288 in a water/propylene glycol aqueous system, where the polymer/aqueous system itself comprises about 30 weight % polymer; about 15 weight % water; and about 55 weight % propylene glycol.

Generally, conventional cosmetic ingredients can be incorporated into the compositions of the invention. However, ingredients that might cause instability or negatively affect the rheology of the product compositions should be present only in amounts that do not cause such difficulties with the composition, and preferably should be avoided. Ingredients can be initially screened for possible incompatibility by admixing the ingredient with the aqueous dispersion of the Boger fluid polymer. For example, it was observed that 10% by weight of ethyl alcohol had a negative effect on the Boger-like rheology of the aqueous polymer system containing 40% water, 20% polymer, and 40% propylene glycol. Accordingly, in one embodiment the compositions of the invention comprise only a low amount of an alkanol, e.g., generally less than about 5%, less than about 3%, preferably less than about 1%, and more preferably less than about 0.1% by weight. In another embodiment the compositions would not contain an alkanol. Alkanols to be avoided are typically C₁ to C₁₂ alcohols, in particular C₁ to C₆ alcohols, and especially ethyl alcohol, propyl alcohol, and butyl alcohol.

It has also been determined, for example that 3% by weight of a polyvinyl alcohol having a pH of about 5-7 and a weight average molecular weight of from about 30,000 to 50,000 also had a negative effect on the rheology of the aqueous polymer system above. Accordingly, in one embodiment the compositions of the invention comprise only a low amount of a polyvinyl alcohol, e.g., generally less than about 5%, less than about 3%, preferably less than about 1%, and more preferably less than about 0.1% by weight. Of course, additional testing using methods known in the cosmetic field, e.g., accelerated phase stability and rheology measurements, should also be performed on the complete formulation to confirm that the formulation has suitable stability and theological properties.

Accordingly, another aspect of the invention relates to methods for imparting a self-leveling property to a mascara. In certain embodiments, the method comprises reducing the alcohol content of a conventional mascara formulation to produce a near alcohol-free composition; and adding an associative thickener comprising a polyurethane polymer as described herein. By “near alcohol-free” is meant that the total alcohol content of the composition will be, e.g., less than about 1%, preferably less than about 0.1%, more preferably less than about 0.01%, and even more preferably less than about 0.001% alcohol by weight, and especially zero percent, based on the total weight of the composition. The percentage of alcohol allowable may depend on the alcohol itself, e.g., ethanol content is preferably less than about 3 weight %; while polyvinyl alcohol content is preferably less than about it weight %. The reduction in alcohol content can avoid, reduce, and/or minimize problems of overly-low viscosity that ensue where an associative thickener described herein is simply added to a convention mascara formulation.

To the near alcohol-free mascara composition, an associative thickener comprising a polyurethane polymer may be added in an amount effective to impart one or more measurable Boger-like properties to the composition. For example, the polyurethane polymer may be added in an amount effective to maintain a constant or near constant viscosity of the near alcohol-free composition over a plurality of shear rates. Preferably, the polyurethane polymer is added in an amount to impart self-leveling properties to the mascara product, as described herein. In some preferred embodiments, a polyurethane polymer is added to give in an amount from about 0.01 weight % to about 30 weight %, from about 0.1 weight % to about 25 weight %, from about 0.5 weight % to about 20 weight %, from about 1 weight % to about 15 weight %, or about 2 to about 10 weight %, based on the total weight of said composition.

The mascara can be, for example, a volumizing mascara, which will typically comprise a high level of wax, typically 10% by weight or greater, or the mascara can be a defining mascara which will typically comprise a low level of wax, usually less than 10% by weight, less than 5% by weight, or even less than 1% by weight wax, and in some instances a defining mascara may be wax-free. In some preferred embodiments, the near-alcohol free composition further comprises a wax, such as rice bran wax, carnauba wax, ouricurry wax, candelilla wax, montan waxes, sugar cane waxes, ozokerite, polyethylene waxes, Fischer-Tropsch waxes, beeswax, botanical waxes, microcrystaline wax, silicone waxes, fluorinated waxes, and any combination thereof, e.g., as discussed above.

In some embodiments, the cosmetic composition comprises pigment dispersions. For example, the cosmetic composition may comprise the pigments Kobo GLW60GBAP and/or Kobo GLW55GBAP. These pigments in certain amounts have been shown to not affect, or to not substantially and/or significantly affect, the Boger-like properties of some compositional embodiments of the instant invention. As discussed above, Kobo GLW60GBAP is a black pigment dispersion while Kobo GLW55 GBAP is a red pigment dispersion, each comprising iron oxides, water, glycerin, ammonium polyacrylate, and methyparaben. An aqueous system comprising about 3 to about 5 weight % of the polyether polyurethane polymer from Rheolate® 288 retains Boger-like properties with the addition of about 0.75 to about 10 weight % Kobo GLW55GBAP and/or Kobo GLW60GBAP. See Example 1, FIG. 1. In some particularly preferred embodiments, a cosmetic composition is provided that comprises about 10 to about 30 weight % of a pigment dispersion and about 2 to about 10 weight % of the polymer from Rheolate® 288 in a water/propylene glycol aqueous system, where the polymer/aqueous system itself comprises about 20 weight % polymer; about 40 weight % water; and about 40 weight % propylene glycol. In other embodiments, the system will comprise about 15 weight % water, about 55 weight % glycol, and about 30 weight % polyurethane polymer. Based on these teachings, one of skill of the art will be able to select other pigments and/or colorants in appropriate amounts to provide additional cosmetic formulations comprising polyether polyurethane polymer and having Boger-like properties, such as self-leveling.

Other colorants may include, for example, organic and inorganic pigments and pearlescent agents. Suitable inorganic pigments include, but are not limited to, titanium oxide, zirconium oxide, and cerium oxide, as well as zinc oxide, iron oxide, chromium oxide, ferric blue, and/or any combinations thereof. Additional colorants include, for example, but are not limited to, D&C Red No. 3, D&C Red No. 6, D&C Red No. 7, D&C Red No. 8, D&C Red No. 9, D&C Red No. 21, D&C Red 22, D&C Red No. 27, D&C Red 28, D&C Red No. 30, D&C Red No. 33, D&C Red 34, D&C Red No. 36, FD&C Red No. 40, D&C Yellow No. 5, FD&C Yellow No. 5, FD&C Yellow No. 6, D&C Yellow No. 10, D&C Green No. 3, D&C Green No. 5, D&C Orange No. 5, FD&C Blue No. 1, annatto, copper powder, bismuth oxychloride, guanine, bronze powder, iron oxides, camel, manganese violet, carmine, mica, titanium dioxide-coated mica, carotene, titanium dioxide, chlorophyllin-calcium complex, and/or any combinations thereof. Suitable organic pigments include barium, strontium, calcium, and aluminium lakes and carbon black. Suitable pearlescent agents include mica coated with titanium oxide, with iron oxide, or with natural pigment.

The composition of various embodiments of the invention may optionally comprise other cosmetic actives and excipients, obvious to those skilled in the art including, but not limited to, fillers, emulsifying agents, antioxidants, surfactants, chelating agents, gelling agents, thickeners, emollients, humectants, moisturizers, vitamins, minerals, viscosity and/or additional rheology modifiers, sunscreens, keratolytics, depigmenting agents, retinoids, hormonal compounds, alpha-hydroxy acids, alpha-keto acids, anti-mycobacterial agents, antifungal agents, antimicrobials, antivirals, analgesics, lipidic compounds, anti-allergenic agents, H1 or H2 antihistamines, anti-inflammatory agents, anti-irritants, antineoplastics, immune system boosting agents, immune system suppressing agents, anti-acne agents, anesthetics, antiseptics, insect repellents, skin cooling compounds, skin protectants, skin penetration enhancers, exfollients, lubricants, fragrances, colorants, depigmenting agents, hypopigmenting agents, preservatives (e.g., DMDM Hydantoin/Iodopropynylbutylcarbonate), stabilizers, pharmaceutical agents, photostabilizing agents, neutralizers triethanolamine) and mixtures thereof.

Thickeners may include, for example, cellulose-based thickeners, for example, water-soluble cellulose-based thickeners, such as hydroxyethylcellulose, methylcellulose, hydroxypropylcellulose and carboxymethylcellulose; gums, for example, gums sold under the name “Cellosize QP 4400 H” by the company Amerchol; guar gum, for example, those sold under the name Vidogum GH 175 by the company Unipectine and under the name Jaguar C by the company Meyhall; quaternized guar gum sold under the name “Jaguar C-13-S” by the company Meyhall; nonionic guar gums comprising C₁-C₆ hydroxyalkyl groups, such as, for example, hydroxymethyl, hydroxyethyl, hydroxypropyl and hydroxybutyl groups, like the guar gums sold under the trade names Jaguar HP8, Jaguar HP60, Jaguar HP120, and Jaguar HP 105 by the company Meyhall, or under the name Galactasol 40H4FD2 by the company Aqualon; xanthan gum, carob gum, scleroglucan gum, gellan gum, rhamsan gum, and karaya gum; alginates, maltodextrin, starch and its derivatives, hyaluronic acid and its salts; clays, for example, montmorillonites, hectorites, and laponites; crosslinked polyacrylic acids, such as the “Carbopol” products from the company Goodrich; the polyglyceryl (meth)acrylate polymers sold under the names “Hispagel” or “Lubragel” by the companies Hispano Quimica or Guardian; polyvinylpyrrolidone; crosslinked acrylamide polymers and copolymers, such as those sold under the names “PAS 5161” or “Bozepol C” by the company Hoechst, or “Sepigel 305” by the company SEPPIC; crosslinked methacryloyloxyethyltrimethylammonium chloride homopolymers sold under the name “Salcare SC95” by the company Allied Colloid; and the like. Based on the teachings herein, a person skilled in the art will be able to select any of these or other optional additives, and/or the amount thereof, such that the desirable properties of the cosmetic compositions described herein are conserved.

The composition can also comprise other ingredients usually used in cosmetics. Such ingredients can be chosen, in particular, from plasticizers, coalescence agents, fillers, dyestuffs, such as pigments or dyes, surfactants, preserving agents, oils, cosmetic agents, such as moisturizers and anti-UV agents that are well known in the art.

Various fillers and additional components may be added. Fillers are normally present in an amount from about 0 weight % to about 20 weight %, based on the total weight of the composition, preferably from about 0.1 weight % to about 10 weight %. Suitable fillers include without limitation silica, treated silica, talc, zinc stearate, mica, kaolin, Nylon powders such as Orgasol™, polyethylene powder, Teflon™, starch such as rich starch, boron nitride, copolymer microspheres such as Expancel™ (Nobel Industries), Polytrap™ (Dow Corning) and silicone resin microbeads (Tospearl™ from Toshiba), polytetrafluoroethylene, and the like. Fillers may be selected to be are compatible with an aqueous medium, where the composition is provided in such, including, in particular the fillers starch, talc and polytetrafluoroethylene. Cosmetic compositions that include an oil phase, e.g., a wax, can use other fillers suitable for non-aqueous systems.

The compositions may have other ingredients such as one or more anesthetics, anti-allergenics, antifungals, anti-inflammatories, antimicrobials, antiseptics, chelating agents, emollients, emulsifiers, fragrances, humectants, lubricants, masking agents, medicaments, moisturizers, pH adjusters, preservatives, protectants, soothing agents, stabilizers, sunscreens, surfactants, thickeners, viscosifiers, vitamins, or any combinations thereof.

In one embodiment, a method of manufacturing a cosmetic composition comprises providing one or more cosmetic ingredients and combining said one or more cosmetic ingredients with an aqueous dispersion of a polyurethane polymer. The aqueous dispersion will comprise water and a polyol. The polyurethane polymer will typically be an associative thickener and, in one embodiment, will result from the reaction of a mixture comprising (i) a polyether polyol component having an average molecular weight between about 500 and about 20,000 g/mol, (ii) a diisocyanate, (iii) a branched, aliphatic C₈₋₃₆ primary alcohol, and (iv) optionally a modifier comprising two groups reactive with said diisocyante and a hydrophobic moiety. In one embodiment, the aqueous dispersion will comprise from about 10-20% by weight water, from about 20-40% by weight of the polyurethane polymer, and from about 50-60% by weight polyol. The polyol may be, without limitation, glycerin or a C₃₋₈ glycol such as propylene butylene glycol, pentylene glycol, hexylene glycol, caprylyl glycol, or the like. In one embodiment, the dispersion will comprise about 15% by weight water, about 30% by weight polyurethane polymer, and about 55% by weight propylene glycol.

### EXAMPLES

### Example 1

### Polyether Polyurethane Aqueous Systems have Boger-Like Properties

Compositions comprising various concentrations of the polyether polyurethane polymer from Rheolate® 288 were prepared and investigated, ranging from 1.0-20.0 weight % solids (polymer) in water. “A” represents a composition comprising 4 weight % polyurethane polymer in water; while “E” represents a composition comprising 5 weight % polyurethane polymer in water. The sample compositions were measured using an AR G2 Rheometer from TA Instruments. Prior to each of the measurements, the sample compositions were loaded and idled for 5 minutes, to allow the sample compositions to reach equilibrium. The sample was then subjected to shear from 1 e⁻⁴ s⁻¹ up to 1,000 as the viscosity was measured. A 40-mm cone and plate geometry, with a 2° cone was used for the measurements. The experiments were conducted at 25° C.

In the concentration range of 1.0 to 20.0 weight %, the composition behaved as a constant viscosity elastic fluid over the shear rate (γ) range of 10⁻⁴-100 s⁻¹. In the case of 4 (composition A) and 5 (composition E) weight % of the polymer, there was a very slight shear thickening (a slight increase in η with increasing γ). That is, there was a very slight shear thickening for the 4 weight % composition over a the shear rate range of from about 10⁻⁴ to about 1.5×10⁻³ s⁻¹; there was a very slight shear thickening for the 5 weight % composition over a shear rate range of from about 10⁻⁴ to about 2.5×10⁻⁴ s⁻¹. Representative results with respect to the 4 and 5 weight % compositions are indicated in FIG. 1.

Additional components were added to the various compositions comprising different concentrations of the polyether polyurethane from Rheolate® 288 to determine whether the added components would affect the Boger-like properties. Constant η behavior was found to be retained for each of the compositions comprising 3, 4 and 5 weight % of the polymer, with the addition of each of: (a) 0.75-10 weight % Kobo GLW60GBAP, a black pigment dispersion, comprising iron oxides, water, glycerin, ammonium polyacrylate, and methyparaben; (h) 0.75-10 weight % Kobo GLW55GBAP, a red pigment dispersion, comprising iron oxides, water, glycerin, ammonium polyacrylate, and methyparaben; (c) 25% of an oil phase composed of 90% Finsolve (C₁₂₋₁₅ Benzoate) and 10% POE (PEG 100 Stearate or PEG 6000 monostearate); and (d) 20% of an oil phase composed of 90% Finsolve (C₁₂₋₁₅ Benzoate) and 10% POE (PEG 100 Stearate or PEG 6000 monostearate). Both (c) and (d) represent emulsions with either (c) 75% or (d) 80% of an aqueous phase comprising the polyether polyurethane polymer from Rheolate® 288, such that the polymer comprises 4 weight % of the total emulsion composition. Representative results with respect to the 4 and 5 weight % compositions with these additional components also are indicated in FIG. 1, where “B” and “F” represent, respectively, a 4 and 5 weight % polyurethane polymer aqueous system, each further comprising 10 weight % Kobo GLW60GBAP, a black pigment dispersion. “C” represents a 4 weight % polyurethane polymer aqueous system further comprising 0.75 weight % Kobo GLW55GBAP, a red pigment dispersion. “D” represents an emulsion having an 80% aqueous phase and a 20% oil phase, where the oil phase comprises 90% Finsolve (C₁₂₋₁₅ Benzoate) and 10% POE (PEG 100 Stearate or PEG 6000 monostearate); and where the aqueous phase comprises the polyether polyurethane polymer from Rheolate® 288, such that the polymer comprises 4 weight % of the total emulsion composition.

The results indicate that the polyurethane polymer from Rheolate® 288 can act as a Boger-like fluid, e.g., retaining its rheological behavior, in aqueous systems, pigment dispersion systems, and in oil/water emulsions, and suggest that the polymer can impart Boger-like properties to cosmetic formulations comprising aqueous systems, pigment dispersion systems, as well as oil-in-water or water-in-oil emulsion systems.

### Example 2

### Mascara Containing Boger Fluid Shows Self-Leveling Properties

A mascara composition comprising polyether polyurethane polymer from Rheolate® 288 (rheolate) was prepared to contain 5 weight % of the polymer and 30 weight % of an iron oxide dispersion in water. Specifically, to 45 g water were added 30 g iron oxides in acrylates copolymer and 25 g of a polymer/aqueous system comprising the polymer from Rheolate® 288 in a water/propylene glycol aqueous system, where the polymer/aqueous system itself comprised 20 weight % polymer; 40 weight % water; and 40 weight % propylene glycol.

The composition was applied to a set of false eyelashes, using 5 strokes of a mascara applicator. The lashes were photographed immediately following initial application of the mascaras (time=0 mins) and again after three minutes (time=3 mins). The results are depicted in FIG. 2.

Observation of the lashes coated with the rheolate with those coated with conventional mascara demonstrates the self-levelling properties of the rheolate. First, the photographs at time=0 mins indicate that the rheolate provides a smooth, uniform coating of the individual lashes even upon initial application. Second, the photographs at time=3 mins show an even more dramatic difference from that expected with conventional mascaras. Any initial clumps on the lashes coated with the mascara composition described herein (rheolate) had smoothed out, to provide a coating having reduced clumping and a smoother surface as compared to the initial coating. The lashes appeared evenly and uniformly coated, providing a clump-free, sleeker, cleaner, and more natural look compared to that obtained with conventional mascaras. These results suggests that compositions described herein can find use as self-leveling, anti-clump mascaras for applying smooth cosmetic films to the eyelashes.

### Example 3

### Exemplary Cosmetic Formulations

Exemplary cosmetic compositions comprising the polyether polyurethane polymer of Rheolate® 288, for use as mascaras, are provided below in Table I (Formulations 1-5). All concentrations are by weight percent of the total composition, unless otherwise stated. Formulation “0” is representative of conventional mascara formulations that lack a self-leveling active taught herein. The compositions each comprises a polymer/aqueous system of 20 weight % polymer, 40 weight % water, and 40 weight % propylene glycol, providing compositions each having 5% polyurethane polymer (as the self-leveling active) based on the total weight of the composition.

TABLE I

| | Formulation | | | | | |
|---|---|---|---|---|---|---|
| | 0 | 1 | 2 | 3 | 4 | 5 |
| Polymer/aq. | 0 | 25 | 25 | 25 | 25 | 25 |
| system (20% | | | | | | |
| polymer, 40% | | | | | | |
| water, 40% | | | | | | |
| propylene | | | | | | |
| glycol) | | | | | | |
| Wax | 24 | 12 | 6 | 3 | 1.5 | 0 |
| Foam Control | 0.2 | 0.2 | 0.2 | 0.2 | 0.2 | 0.2 |
| Agent | | | | | | |
| Gum Arabic | 0.2 | 0 | 0 | 0 | 0 | 0 |
| Hydroxyethyl | 0.2 | 0 | 0 | 0 | 0 | 0 |
| Cellulose | | | | | | |
| Paraben | 0.8 | 0.8 | 0.8 | 0.8 | 0.8 | 0.8 |
| Poly- | 0.2 | 0.2 | 0.2 | 0.2 | 0.2 | 0.2 |
| quaternium-10 | | | | | | |
| Sodium | 0.3 | 0.3 | 0.3 | 0.3 | 0.3 | 0.3 |
| Hexameta- | | | | | | |
| phosphate | | | | | | |
| Tetrasodium | 0.1 | 0.1 | 0.1 | 0.1 | 0.1 | 0.1 |
| EDTA | | | | | | |
| Triethanol- | 2.1 | 2.1 | 2.1 | 2.1 | 2.1 | 2.1 |
| amine | | | | | | |
| Iron Oxides | 7 | 7 | 7 | 7 | 7 | 7 |
| VP/Eicosene | 2 | 2 | 2 | 2 | 2 | 2 |
| Copolymer | | | | | | |
| Stearic Acid | 5.5 | 5.5 | 5.5 | 5.5 | 5.5 | 4.1 |
| Glyceryl | 0.7 | 0.7 | 0.7 | 0.7 | 0.7 | 0.7 |
| Monostearate | | | | | | |
| Filler | 3.1 | 3.1 | 3.1 | 3.1 | 3.1 | 3.1 |
| Polybutene | 1.2 | 1.2 | 1.2 | 1.2 | 1.2 | 1.2 |
| Emollient | 0.5 | 0.5 | 0.5 | 0.5 | 0.5 | 0.5 |
| Poly- | 0.3 | 0.3 | 0.3 | 0.3 | 0.3 | 0.3 |
| quaternium-7 | | | | | | |
| Preservative | 0.35 | 0.35 | 0.35 | 0.35 | 0.35 | 0.35 |
| Film Former. | 0.2 | 0.2 | 0.2 | 0.2 | 0.2 | 0.2 |
| Demineralized | qs | qs | qs | qs | qs | qs |
| Water | | | | | | |
| Total | 100 | 100 | 100 | 100 | 100 | 100 |

### Example 4

### Exemplary Cosmetic Formulation

Another exemplary cosmetic composition comprising the polyether polyurethane polymer of Rheolate® 288, for use as mascaras, is provided below in table II (Formulation 6). All concentrations are by weight percent of the total composition, unless otherwise stated. The composition comprises a polymer/aqueous system of 30 weight polymer, 15 weight % water, and 55 weight % propylene glycol, providing a composition having 2.5% polyurethane polymer as the self-leveling active.

TABLE II

| Formulation | 6 |
|---|---|
| Polymer/aq. system (30% polyurethane, 15% water, | 8.4 |
| 55% propylene glycol) | |
| Wax | 12 |
| Foam Control Agent | 0.2 |
| Triethanolamine | 1.05 |
| Stearic Acid | 2.75 |
| Paraben | 0.8 |
| Polyquaternium-10 | 0.2 |
| Sodium Hexametaphosphate | 0.3 |
| Tetrasodium EDTA | 0.1 |
| Iron Oxides | 7 |
| VP/Eicosene Copolymer | 2 |
| Glyceryl Monostearate | 0.7 |
| Filler | 3.1 |
| Polyisobutene | 1.2 |
| Emollient | 0.5 |
| Polyquaternium-7 | 0.3 |
| Preservative | 0.35 |
| Demineralized Water | qs |
| Total | 100 |

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method for applying a smooth cosmetic film to eyelashes, comprising:
forming an initial coating on said eyelashes with a composition comprising by weight of the composition from about 0.01 to about 30% by weight of an associative thickener capable of forming a film on the shaft of a keratin fiber; and allowing the coating to self-level to provide a coating having reduced clumping and a smoother surface as compared to said initial coating;
wherein said associative thickener comprises a polyurethane polymer resulting from the reaction of a mixture comprising (i) a polyether polyol component having an average molecular weight between about 500 and about 20,000 g/mol, (ii) a diisocyanate, (iii) a branched, aliphatic C₈₋₃₆ primary alcohol, and (iv) optionally a modifier comprising two groups reactive with said diisocyante and a hydrophobic moiety.

2. The method according to claim 1 wherein said polyurethane polymer comprises polyether urea polyurethane polymer.

3. The method according to claim 1 wherein said composition further comprises less than 0.1% alcohol by weight.

4. The method according to claim 1 wherein said composition further comprises a wax.

5. The method according to claim wherein said composition further comprises an aqueous vehicle.

6. The method according to claim 5 wherein said aqueous vehicle comprises propylene glycol.

7. The method according to claim 1 wherein said composition comprises further an oil-in-water or a water-in-oil emulsion.

8. (canceled)

9. The method according to claim 1 wherein said polymer is in an amount from about 0.01 to about 10% by weight based on the total weight of the composition.

10. The method according to claim 1 wherein said composition is a mascara.

11. A method for imparting a self-leveling property to a mascara composition, comprising:
formulating said mascara to comprise an associative thickener comprising by weight of the mascara composition from about 0.01 to about 30% by weight of a polyurethane polymer resulting from the reaction of a mixture comprising (i) a polyether polyol component having an average molecular weight between about 500 and about 20,000 g/mol, (ii) a diisocyanate, (iii) a branched, aliphatic C₈₋₃₆ primary alcohol, and (iv) optionally a modifier comprising two groups reactive with said diisocyante and a hydrophobic moiety; and
said mascara containing an amount of alcohol such that the mascara is a near alcohol-free composition;
wherein said polyurethane polymer is added in an amount effective to maintain a constant or near constant viscosity of said near alcohol-free composition over a plurality of shear rates.

11. The method according to claim 10 wherein said polyurethane polymer comprises a polyether urea polyurethane polymer.

13. (canceled)

14. The method according to claim 10 wherein said polymer is in an amount from 2 weight % to 10 weight % based on the total weight of said composition.

15. The method according to claim 11 wherein said mascara further comprises a wax.

16. The method according to claim 11 wherein said composition comprises an oil-in-water or a water-in-oil emulsion, and wherein said aqueous emulsion comprises propylene glycol.

17. (canceled)

18. A method of manufacturing a cosmetic composition comprising incorporating into said cosmetic composition a dispersion in an aqueous polyol of a polyurethane polymer resulting from the reaction of a mixture comprising (i) a polyether polyol component having an average molecular weight between about 500 and about 20,000 g/mol, (ii) a diisocyanate, (iii) a branched, aliphatic C8-36 primary alcohol, and (iv) optionally a modifier comprising two groups reactive with said diisocyanate and a hydrophobic moiety.

19. The method according to claim 13, wherein said dispersion comprises from about 10% to about 60% by weight water, from about 10% to about 40% by weight polyurethane polymer, and from about 10% to about 70% by weight polyol.

20. The method according to claim 14, wherein said polyol is selected from glycerin or a C₃₋₈ glycol, or combinations thereof.

21. The method according to claim 15, wherein said polyol comprises propylene.

22. The method according to claim 14, wherein said dispersion comprises about 15% by weight water, about 30% by weight polyurethane polymer, and about 55% by weight propylene glycol.

23. The method according to claim 14, wherein said dispersion comprises about 40% by weight water, about 20% by weight polyurethane polymer, and about 10% by weight propylene glycol.
